(19) United States
(12) Patent Application Publication
(10) Pub. No.: US 2026/0028121 A1
(43) Pub. Date: Jan. 29, 2026

(54) FACILITATING CIRCADIAN COMFORT IN A SMART AIRCRAFT ENVIRONMENT

(71) Applicant: GOODRICH LIGHTING SYSTEMS, INC., Phoenix, AZ (US)

(72) Inventor: Sreekanth Yellavula, Hyderabad (IN)

(73) Assignee: GOODRICH LIGHTING SYSTEMS, INC., Phoenix, AZ (US)

(21) Appl. No.: 19/219,656

(22) Filed: May 27, 2025

(30) Foreign Application Priority Data
Jul. 23, 2024 (IN) 202441056075

(51) Int. Cl. B64D 11/00; A61M 21/02; B64D 13/06; B64D 45/00

(52) U.S. Cl. CPC B64D 11/00; A61M 21/02; B64D 13/06; B64D 45/00; A61M 2205/3303; B64D 2013/0655

(57) ABSTRACT

A suite management system is provided. The suite management system includes a controller. The controller is configured to obtain passenger preferences from a passenger occupying a suite, obtain circadian data from the passenger, analyze the circadian data to identify one or more changes to the suite to align with the passenger preferences, and, responsive to identifying the one or more changes, automatically adjusting one or more electronic devices within the suite to align with the passenger preferences.

### CROSS-REFERENCE TO RELATED APPLICATIONS

This application claims priority to, and the benefit of, India Provisional Patent Application No. 202441056075, filed Jul. 23, 2024 (DAS Code CF17) and titled “FACILITATING CIRCADIAN COMFORT IN A SMART AIRCRAFT ENVIRONMENT,” which is incorporated by reference herein in its entirety for all purposes.

### FIELD

The present disclosure generally relates to control systems and more particularly to facilitating circadian comfort in a smart aircraft environment for use within a luxury or comfort suite on an aircraft.

### BACKGROUND

In luxury or comfort suites on an aircraft, suite management systems offer one or more of adjustable seating, entertainment, climate control, and lighting, which are set and adjustable by the passenger(s) occupying the luxury or comfort suite.

### SUMMARY

Disclosed herein is a suite management system. The suite management system includes a controller. The controller is configured to obtain passenger preferences from a passenger occupying a suite, obtain circadian data from the passenger, analyze the circadian data to identify one or more changes to the suite to align with the passenger preferences, and, responsive to identifying the one or more changes, automatically adjust one or more electronic devices within the suite to align with the passenger preferences.

In various embodiments, the circadian data comprises at least one of a heart rate, body temperature, sleep patterns, blood oxygen saturation levels, or a heart rate variability (HRV) of the passenger.

In various embodiments, the circadian data further comprises at least one of respiratory rate, skin temperature gradient, electrodermal activity, cognitive load, gastrointestinal activity, hydration, mood and emotional state, movement, activity level, sleep quality, allergy and air quality, thermal comfort, blood glucose, A1C, posture, pressure points, vibration or balance.

In various embodiments, the circadian data is obtained from at least one of a smartwatch, a fitness tracker, an activity tracker, or a phone of the passenger.

In various embodiments, the circadian data is obtained at least one sensor coupled to the at least one of the smartwatch, the fitness tracker, the activity tracker, or the phone of the passenger.

In various embodiments, the at least one sensor comprises at least one of a respiratory sensor, a heart rate sensor, a skin temperature sensor, a electrodermal activity sensor, a smart patch or ingestible sensor, an electroencephalogram (EEG) sensor, a voice analysis sensor, a facial expression sensor, a hydration tracking sensor, an oxygen saturation (SpO₂) sensor, an environmental sensor, a continuous glucose monitoring (GCM) sensor, an A1C sensor, a posture sensor, a pressure point mapping sensor, an accelerometer sensor, or a gyroscope sensor.

In various embodiments, the one or more electronic devices comprise at least one of a light, a seat, a temperature control system, a flight attendant call system, or an in-flight entertainment system.

In various embodiments, the one or more electronic devices further comprise at least one of an oxygen supply system, an air circulation system, a humidity control system, an air purifier, or an air filtration system.

In various embodiments, the controller is at least one of electronically or communicatively coupled to an electrical system or a communication system in order to adjust the one or more electronic devices within the suite.

In various embodiments, the controller is further configured to analyze the circadian data to identify at least one of a suggestion, a reminder, or an alert send to the passenger and, responsive to identifying the at least one of the suggestion, the reminder, or the alert, automatically send the at least one of the suggestion, the reminder, or the alert to the passenger.

Also disclosed herein is an aircraft. The aircraft including a suite and a suite management system deployed within the suite. The suite management system includes a controller. The controller is configured to obtain passenger preferences from a passenger occupying a suite, obtain circadian data from the passenger, analyze the circadian data to identify one or more changes to the suite to align with the passenger preferences, and, responsive to identifying the one or more changes, automatically adjust one or more electronic devices within the suite to align with the passenger preferences.

In various embodiments, the circadian data comprises at least one of a heart rate, body temperature, sleep patterns, blood oxygen saturation levels, or a heart rate variability (HRV) of the passenger.

In various embodiments, the circadian data further comprises at least one of respiratory rate, skin temperature gradient, electrodermal activity, cognitive load, gastrointestinal activity, hydration, mood and emotional state, movement, activity level, sleep quality, allergy and air quality, thermal comfort, blood glucose, A1C, posture, pressure points, vibration or balance.

In various embodiments, the circadian data is obtained from at least one of a smartwatch, a fitness tracker, an activity tracker, or a phone of the passenger.

In various embodiments, the circadian data is obtained at least one sensor coupled to the at least one of the smartwatch, the fitness tracker, the activity tracker, or the phone of the passenger.

In various embodiments, the at least one sensor comprises at least one of a respiratory sensor, a heart rate sensor, a skin temperature sensor, a electrodermal activity sensor, a smart patch or ingestible sensor, an electroencephalogram (EEG) sensor, a voice analysis sensor, a facial expression sensor, a hydration tracking sensor, an oxygen saturation (SpO₂) sensor, an environmental sensor, a continuous glucose monitoring (GCM) sensor, an A1C sensor, a posture sensor, a pressure point mapping sensor, an accelerometer sensor, or a gyroscope sensor.

In various embodiments, the one or more electronic devices comprise at least one of a light, a seat, a temperature control system, a flight attendant call system, or an in-flight entertainment system.

In various embodiments, the one or more electronic devices further comprise at least one of an oxygen supply system, an air circulation system, a humidity control system, an air purifier, or an air filtration system.

In various embodiments, the controller is at least one of electronically or communicatively coupled to an electrical system or a communication system in order to adjust the one or more electronic devices within the suite.

In various embodiments, the controller is further configured to analyze the circadian data to identify at least one of a suggestion, a reminder, or an alert send to the passenger and, responsive to identifying the at least one of the suggestion, the reminder, or the alert, automatically send the at least one of the suggestion, the reminder, or the alert to the passenger.

The foregoing features and elements may be combined in any combination, without exclusivity, unless expressly indicated herein otherwise. These features and elements as well as the operation of the disclosed embodiments will become more apparent in light of the following description and accompanying drawings.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the following detailed description and claims in connection with the following drawings. While the drawings illustrate various embodiments employing the principles described herein, the drawings do not limit the scope of the claims.

FIG. 1 illustrates a suite, in accordance with various embodiments.

FIG. 2 illustrates a view from inside suite, in accordance with various embodiments.

FIG. 3 illustrates a suite management system, in accordance with various embodiments.

FIG. 4 illustrates mechanisms within a data collection mechanism, in accordance with various embodiments.

FIG. 5 illustrates mechanisms within a data transmission mechanism, in accordance with various embodiments.

FIG. 6 illustrates mechanisms within a data analysis mechanism, in accordance with various embodiments.

FIG. 7 illustrates mechanisms within a system integration mechanism, in accordance with various embodiments.

FIG. 8 illustrates mechanisms within an autonomous adjustments mechanism, in accordance with various embodiments.

FIG. 9 illustrates a method for adjusting suite conditions based on circadian rhythms of a passenger, in accordance with various embodiments.

### DETAILED DESCRIPTION

The following detailed description of various embodiments herein makes reference to the accompanying drawings, which show various embodiments by way of illustration. While these various embodiments are described in sufficient detail to enable those skilled in the art to practice the disclosure, it should be understood that other embodiments may be realized and that changes may be made without departing from the scope of the disclosure. Thus, the detailed description herein is presented for purposes of illustration only and not of limitation. While these exemplary embodiments are described in sufficient detail to enable those skilled in the art to practice the invention, it should be understood that other embodiments may be realized and that logical, chemical and mechanical changes may be made without departing from the spirit and scope of the invention. For example, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected, or the like may include permanent, removable, temporary, partial, full or any other possible attachment option. Additionally, any reference to without contact (or similar phrases) may also include reduced contact or minimal contact. It should also be understood that unless specifically stated otherwise, references to “a,” “an” or “the” may include one or more than one and that reference to an item in the singular may also include the item in the plural. Further, all ranges may include upper and lower values and all ranges and ratio limits disclosed herein may be combined.

As stated previously, in luxury or comfort suites on an aircraft, suite management systems offer one or more of adjustable seating, entertainment, climate control, and lighting, which are set and adjustable by the passenger(s) occupying the luxury or comfort suite.

While such suite management systems offer adjustable seating, entertainment, climate control, and lighting, typical suite management systems neglect a passenger's circadian rhythms and lack techniques for real-time adjustments. Circadian rhythms regulate vital body functions like sleep, hormones, and/or metabolism, among others. Aligning these circadian rhythms with local time is crucial to avoid jet lag during long-haul flights. However, frequent air travel may disrupt this alignment, leading to discomfort and affecting sleep, mood, and/or hydration, among others, thereby leading to one or more of disrupted sleep, increased fatigue, and/or difficulty adapting to new time zones, which may exacerbate jet lag and delay recovery. In order to address such issues, suite conditions should be adjusted to match the passenger's circadian rhythms, an aspect overlooked by typical cabin systems and lighting solutions.

Disclosed herein is a suite management system for establishing a topology on using a passenger's personal devices, combined with their expressed desire of time zone adaption to adjust conditions, i.e. seating, entertainment, climate control, and/or lighting, among others, within the suite occupied by the passenger. In various embodiments, the suite management system utilizes voluntary access to a passenger's personal devices, such as smartwatches, fitness tracker, activity tracker, and/or phone, among others, and incorporates this data to regulate the cabin or suite, i.e. the passenger's personal space, to allow the passenger to tailor their experience for maximum time zone adjustment. In that regard, in various embodiments, the passenger is prompted to share personal data related to biological functions from their personal devices together with their expressed desire of adjustment. In various embodiments, responsive to the passenger consenting to share the person data, a controller within the suite management system is configured to establish a connection to the personal device via a connection service, such as IEEE 802.11 interface (Wi-Fi), an IEEE 802.15.1 interface (Bluetooth), and/or an IEEE 802.15.4 interface (ZigBee), among others. In various embodiments, the controller is configured to collect the passenger's circadian information together with the passenger's expressed desire of adaptation of the conditions within the suite. In various embodiments, the controller is configured to be communicatively coupled to a suite management system that may be adjusted to the passenger's person space, i.e. seating, entertainment, climate control, and/or lighting, among others. In that regard, in various embodiments, responsive to the controller detecting, based on the gathered circadian information from the personal device data, the passenger falling asleep, the controller is configured to maximize the passenger's sleep by controlling one or more of the adjustable features, such as one or more of dimming the lights, reducing blue light, stopping in-flight entertainment, producing calming sounds, informing a flight crew not to provide service for the duration of the passenger's sleep, raising or lowering a suite temperature, among others. While the control may perform certain autonomous controls while the passenger is asleep, the controller may perform some of the same autonomous operation while the passenger is awake as well as other autonomous operations, such as recommending breaks, scheduling meal timing, sending notification to drink water, among others. Therefore, the disclosed suite management system utilizes data shared from the passenger's personal devices along with the passenger's expressed desire of adaptation of the conditions within the suite to gather the data, define specific actions to control the passenger's personal space, and provide directions to the flight crew in order to maximize the passenger's chronobiological choices and reduce jet lag.

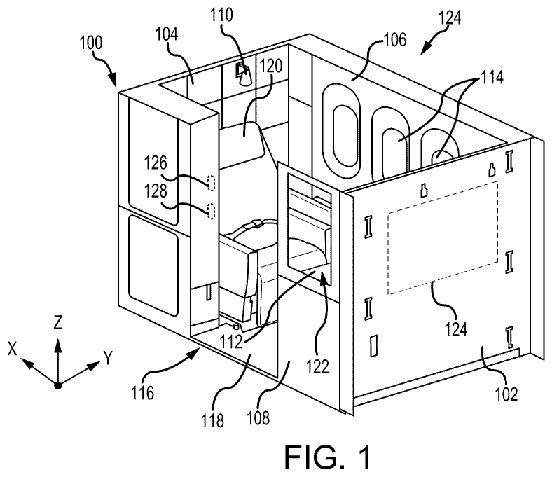

Referring now to FIG. 1, in accordance with various embodiments, a suite 100 is illustrated. In various embodiments, the suite 100 may include a forward wall 102, an aft wall 104, an exterior wall 106, an interior wall 108, a light 110, a surface 112, one or more windows 114 disposed in exterior wall 106, a doorway 116 disposed in interior wall 108, a floor 118, and a seat 120. In various embodiments, suite 100 may further include an interior window 122 in interior wall 108. In various embodiments, the suite 100 may further include an in-flight entertainment (IFE) system 124, a temperature control system 126, and/or a flight attendant call system 128 located in the suite 100.

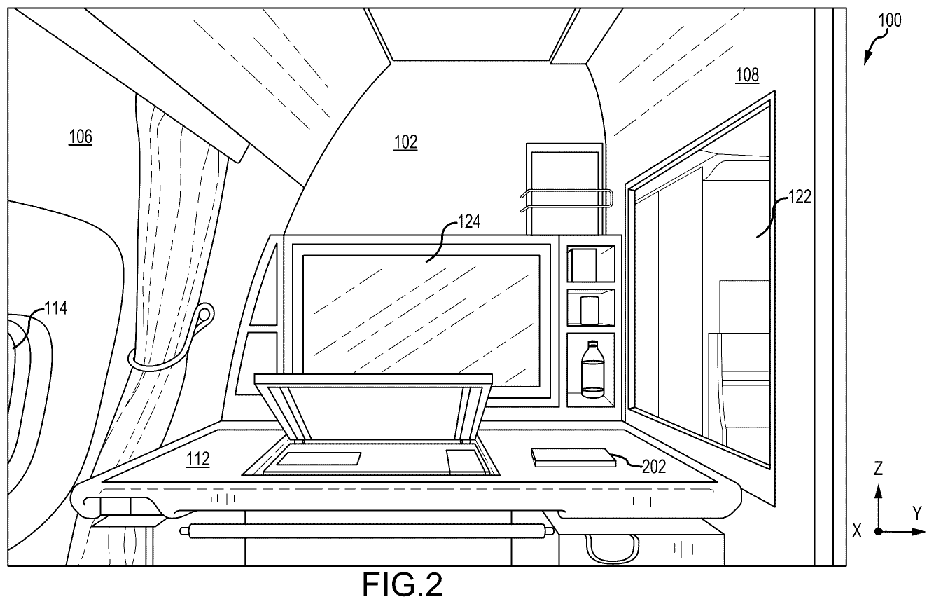

Referring now to FIG. 2, in accordance with various embodiments, a view from inside suite 100 is illustrated. With reference to the components of suite 100 described in FIG. 1, the view illustrated in FIG. 2 depicts that the suite 100 may include the forward wall 102, an exterior wall 106, an interior wall 108, a surface 112, one or more windows 114 disposed in exterior wall 106, an interior window 122 in interior wall 108, and the IFE system 124. Additionally, in various embodiments, the suite 100 may include suite management system 202. In various embodiments, the suite management system 202 is configured to automatically control, under command of a passenger, various electronic equipment, such as controlling the light 110, positioning of the seat 120, setting a temperature of the suite 100 via the temperature control system 126, and/or calling/notifying a flight attendant via the flight attendant call system 128 of FIG. 1, and/or controlling the IFE system 124 of FIGS. 1 and 2, based on circadian data shared from the passenger's personal devices along with the passenger's expressed desire of adaptation of the conditions within the suite. In that regard, in various embodiments, the suite management system 202 is configured with electronic circuitry to connect to the fixed base and electrically and/or communicatively interface with various other services within the aircraft, via a power system, IEEE 802.11 interface (Wi-Fi), an IEEE 802.3 interface (ethernet), an IEEE 802.15.1 interface (Bluetooth), and/or an IEEE 802.15.4 interface (ZigBee), among others. In various embodiments, by electrically and/or communicatively interfacing with various other services within the aircraft, the suite management system is configured to control the lights, position of the seat, a temperature of the suite, call/notification to flight attendant, and/or the entertainment system, among others.

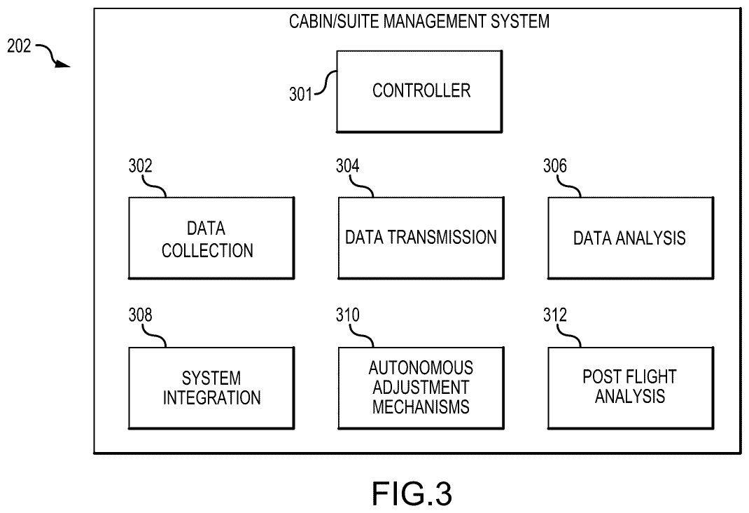

Referring now to FIG. 3, in accordance with various embodiments, a suite management system is illustrated. In various embodiments, the suite management system 202 includes a controller 301 in communication with a data collection mechanism 302, a data transmission mechanism 304, a data analysis mechanism 306, a system integration mechanism 308, autonomous adjustment mechanisms 310, and post-flight analysis mechanism 312. The controller 301 may include a logic device such as one or more of a central processing unit (CPU), an accelerated processing unit (APU), a digital signal processor (DSP), a field programmable gate array (FPGA), an application specific integrated circuit (ASIC), or the like. In various embodiments, the controller 301 may further include any non-transitory memory known in the art. The memory may store instructions usable by the logic device to perform operations as described herein.

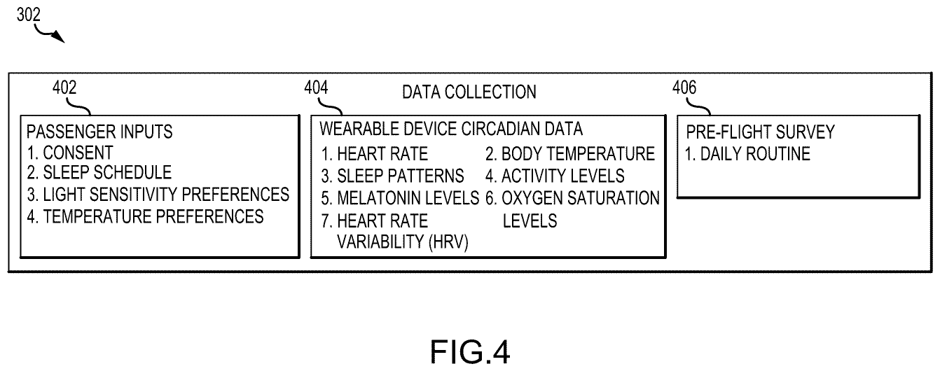

With further reference to FIG. 4 that, in accordance with the various embodiments, illustrates mechanisms within the data collection mechanism 302, in various embodiments, a passenger input collection mechanism 402 may be configured to obtain the passenger's consent for sharing personal biological data for the flight in order to control the lights, position of the seat, a temperature of the suite, call/notification to flight attendant, and/or the entertainment system, among others, while the passenger sleeps. In various embodiments, the passenger input collection mechanism 402 may present information to the passenger to ensure that the passenger is well informed about how their data will be used specifically for controlling the lights, position of the seat, a temperature of the suite, call/notification to flight attendant, and/or the entertainment system, among others, while the passenger sleeps and allows the passenger to manage the passenger's personal preferences. In various embodiments, passenger input collection mechanism 402 may require that the passenger agree to synchronize their circadian data or controlling the features of the smart cabin environment on the flight. Once the consent is granted by the passenger, the data collection mechanism 304 may be configured to gather passenger data.

In that regard, in various embodiments, the passenger input collection mechanism 402 may be configured to collect information pertaining to one or more of the passenger's preferred sleep schedule, the passenger's light sensitivity preferences, or the passenger's temperature preferences, among others. In various embodiments, the wearable device circadian data collection mechanism 404 may be configured to collect circadian data related to one or more of the passenger's heart rate, body temperature, sleep patterns, blood oxygen saturation levels, or heart rate variability (HRV), among others, collected via various sensors within or coupled to the passenger's smartwatch, fitness tracker, activity tracker, and/or phone, among others. The circadian information may further include one or more of respiratory rate, skin temperature gradient, electrodermal activity, cognitive load, gastrointestinal activity, hydration, mood and emotional state, movement and activity levels, sleep quality, allergy and air quality, thermal comfort, blood glucose, A1C, posture, pressure points, or vibration and balance, among others, collected via various sensors within or coupled to the passenger's smartwatch, fitness tracker, activity tracker, and/or phone, among others. In various embodiments, the various sensors may include one or more of a respiratory sensor, a heart rate sensor, a skin temperature sensor, a electrodermal activity sensor, a smart patch or ingestible sensor, an electroencephalogram (EEG) sensor, a voice analysis sensor, a facial expression sensor, a hydration tracking sensor, an oxygen saturation (SpO₂) sensor, an environmental sensor, a continuous glucose monitoring (GCM) sensor, an A1C sensor, a posture sensor, a pressure point mapping sensor, or an accelerometer and/or gyroscope sensor, coupled to or within the passenger's smartwatch, fitness tracker, activity tracker, and/or phone. In various embodiments, the pre-flight survey mechanism 406 may be configured to survey the passenger to gather information pertaining to the passenger's daily routine.

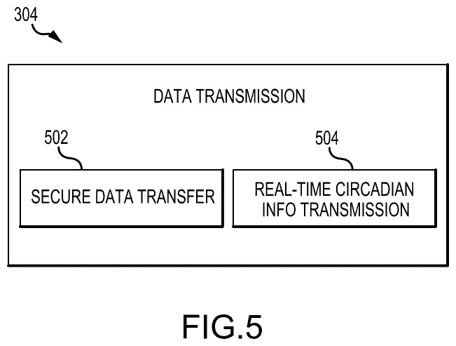

Returning to FIG. 3 and with further reference to FIG. 5 that, in accordance with the various embodiments, illustrates mechanisms within the data transmission mechanism 304, in various embodiments, the data transmission mechanism 304 may be configured to provide communication connectivity via secure data transfer mechanism 502, such as IEEE 802.11 interface (Wi-Fi), an IEEE 802.15.1 interface (Bluetooth), and/or an IEEE 802.15.4 interface (ZigBee), among others. In that regard, the data transmission mechanism 304 is configured to provide for a transfer passenger data from the passenger's smartwatch, fitness tracker, activity tracker, and/or phone, among others, to the suite management system 202. In various embodiments, real-time circadian information transmission mechanism 504 may be configured to receive and transmit the circadian information collected via the data collection mechanism 302. In various embodiments, the data transmission mechanism 304 may transmit the passenger's preferred sleep schedule, the passenger's light sensitivity preferences, the passenger's temperature preferences, the circadian data, as well as the passenger's survey answers received by the real-time circadian information transmission mechanism 504 from the data collection mechanism 302, via the secure data transfer mechanism 502 to the data analysis mechanism 306. In various embodiments, the data transmission mechanism 304 may be configured to continuously transmit further circadian data before, during, and/or after a flight, as long as the passenger grants connectivity to the data transmission mechanism 302.

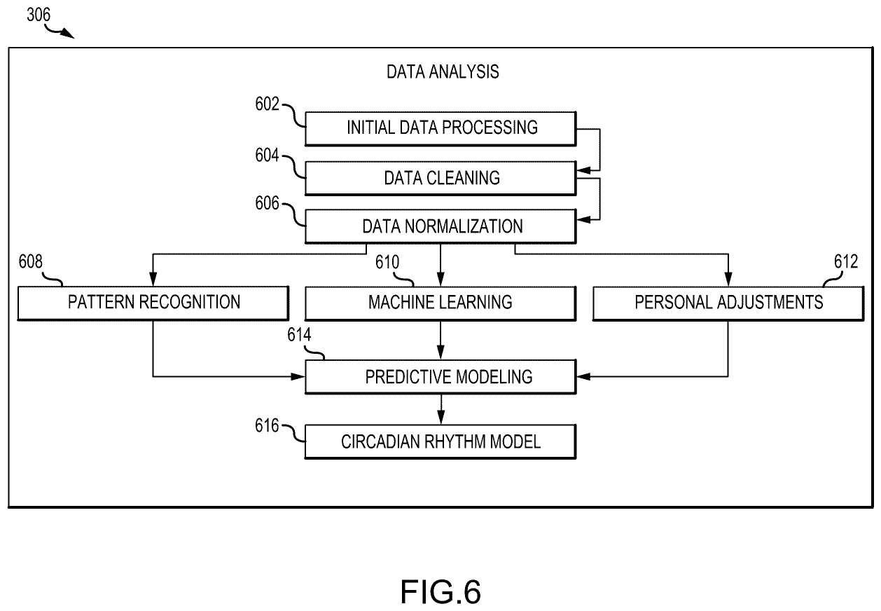

Returning to FIG. 3 and with further reference to FIG. 6 that, in accordance with the various embodiments, illustrates mechanisms within the data analysis mechanism 306, in various embodiments, the data analysis mechanism 306 may be configured to perform initial data processing 602 on the passenger's preferred sleep schedule, the passenger's light sensitivity preferences, the passenger's temperature preferences, the circadian data, as well as the passenger's survey answers to identify key aspects in the received data that will aid in controlling the lights, position of the seat, a temperature of the suite, call/notification to flight attendant, and/or the entertainment system, among others, while the passenger sleeps. In various embodiments, the data analysis mechanism 306 may be configured to perform data cleaning 604 to remove superfluous information and preform data normalization 606 to organize data entries to ensure they appear similar across all fields and records. In various embodiments, the data analysis mechanism 306 may be configured to perform one or more of pattern recognition 608, machine learning 610, or identify personal adjustments 612 in order to generate predictive modeling 614 so as to identify a circadian rhythm model 616 for the passenger.

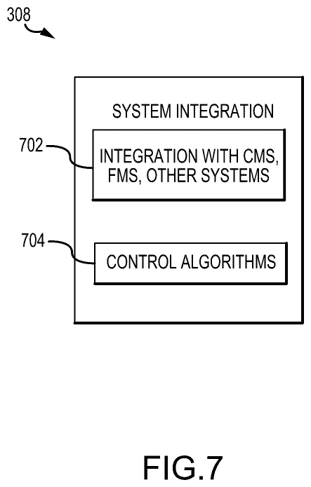

Returning to FIG. 3 and with further reference to FIG. 7 that, in accordance with the various embodiments, illustrates mechanisms within system integration mechanism 308, in various embodiments, the system integration mechanism 308 includes integration mechanisms 702 for integration with one or more of cabin management systems (CMS) or flight management systems (FMS), among others, and configured for controlling the lights, position of the seat, a temperature of the suite, call/notification to flight attendant, and/or the entertainment system, among others. In various embodiments, the system integration mechanism 308 further includes control algorithms 704 that are utilized to control the lights, position of the seat, a temperature of the suite, call/notification to flight attendant, and/or the entertainment system, among others, based on the collected circadian data collected by the data collection mechanism 304 and using the circadian rhythm model 616 of the data analysis mechanism 306.

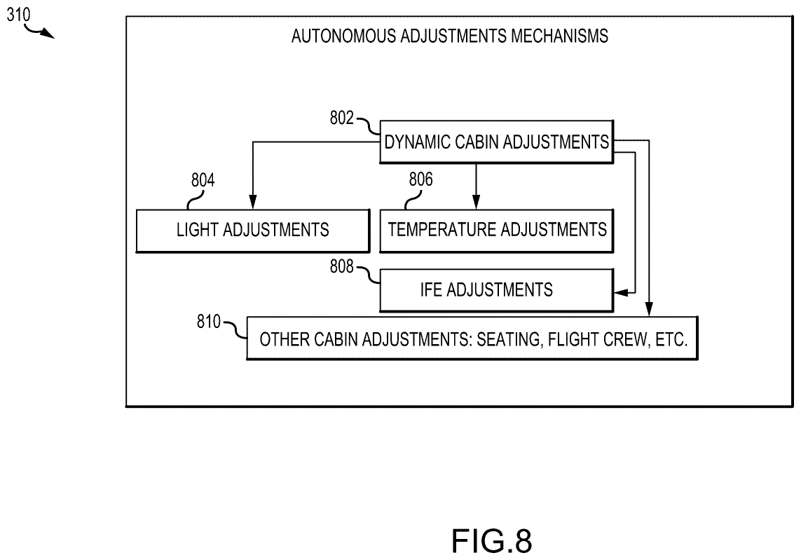

Returning to FIG. 3 and with further reference to FIG. 8 that, in accordance with the various embodiments, illustrates mechanisms within the autonomous adjustments mechanism 310, in various embodiments, the autonomous adjustments mechanism 310 includes dynamic cabin adjustments 802. In various embodiments, based on information from the control algorithms 704 of FIG. 7, dynamic cabin adjustments 806 may implement various light adjustments 804, such as dimming the lights to reduce blue light exposure during high-stress periods to create a calming environment, adjusting cabin lighting to improve the passenger's mood, or using bright, cool lighting tones during periods of high cognitive demand and warm, dim tones during rest phases to support mental recovery, gradually increase light intensity to gently wake the passenger during light sleep stages to reduce grogginess, among others.

In various embodiments, based on information from the control algorithms 704 of FIG. 7, dynamic cabin adjustments 806 may implement various temperature adjustments, such as adjusting cabin temperature during the passenger's sleep to create an optimal sleep environment. In various embodiments, based on information from the control algorithms 704 of FIG. 7, dynamic cabin adjustments 806 may implement in-flight entertainment (IFE) adjustments 808, such as playing soothing background music to reduce stress, recommending relaxing or engaging content based on emotional state, playing calming, rhythmic sounds that match slow breathing patterns to enhance sleep quality, or providing content suggestions (movies, music, games) based on current emotional state, among others.

In various embodiments, based on information from the control algorithms 704 of FIG. 7, dynamic cabin adjustments 806 may implement other cabin adjustments 810, such as adjusting seat climate controls to provide localized cooling or warming based on skin temperature gradients, promoting comfort during sleep and wake phases, adjusting seat position to encourage movement during long periods of inactivity or provide support during relaxation phases, adjusting seat heating or cooling functions to maintain optimal comfort, adjust seat position to improve the passenger's posture thereby reducing discomfort and preventing back pain, which may include dynamically adjusting seat support features (e.g., lumbar support, headrest position, etc.) based on posture data, adjusting seat cushions to redistribute pressure and enhance comfort, particularly during long flights, activate seat massage features when increased pressure is detected in specific areas of the passenger's body, adjusting seat stabilization systems use real-time balance and vibration data from the flight management system to minimize discomfort during turbulence, among others. In various embodiments, the other cabin adjustments 810 may further include increasing oxygen levels via an oxygen supply system and/or optimizing air circulation via an air circulation system during deep sleep phases to support restful sleep, increasing cabin humidity via a humidity control system to maintain hydration levels, particularly during long flights, or activating air purifiers via an air purifier or adjust filtration via an air filtration system settings when allergen levels are high, among others. In various embodiments, the other cabin adjustments 810 may include sending suggestions, reminders, and/or alerts to the passenger, such as sending reminders to drink water when temperature gradients suggest dehydration, suggesting cognitive breaks or naps when high theta wave activity indicates mental fatigue, suggest light exercises or stretches to reduce discomfort during long flights, alerting the passenger to changes in air quality and suggest preventive measures (e.g., wearing a mask or taking allergy medication, among others), suggesting healthy snacks at optimal times to prevent blood sugar dips and spikes, improving overall passenger comfort and energy levels, or suggesting balance exercises or provide supportive seating arrangements to improve stability and comfort, among others. In various embodiments, the other cabin adjustments 810 may include scheduling meal service to align with high digestive activity phases for better digestion, suggesting easy-to-digest options during low digestive activity phases or more substantial meals during peak digestive times, or suggesting personalized meal options to maintain stable blood glucose levels or A1C levels, among others.

Returning to FIG. 3, in various embodiments the post-flight analysis mechanism 312 may be configured to review the collected data to assess the effectiveness of the adjustments made during the flight. In various embodiments, the post-flight analysis mechanism 312 may analyze passenger feedback, circadian data, and other relevant parameters to evaluate the overall comfort level and effectiveness of the system in mitigating jet lag and enhancing passenger well-being. In various embodiments, the analysis may assist in identifying areas for improvement and refine the system for future flights. In various embodiments, the post-flight analysis mechanism 312 may further survey the passenger at the end of the flight for passenger feedback, which may be utilized in the post-flight analysis.

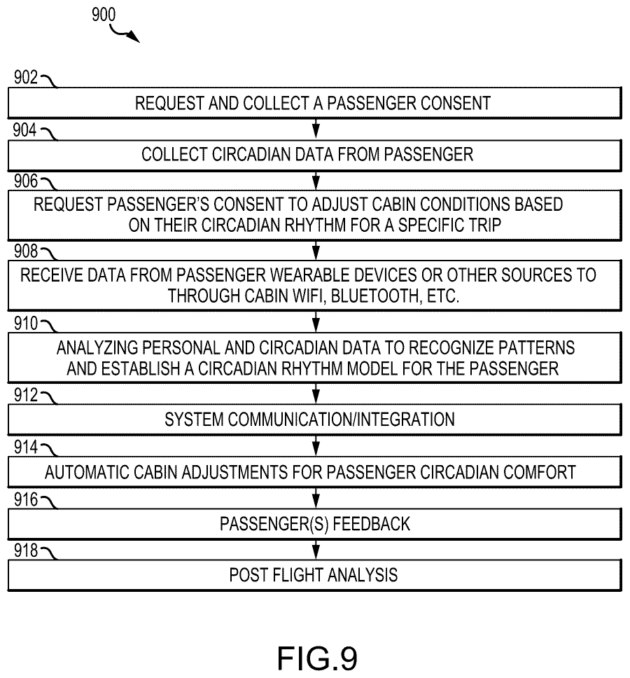

Referring now to FIG. 9, in accordance with various embodiments, a method 900 for adjusting suite conditions based on circadian rhythms of a passenger, is illustrated. The method 900 may be performed by a controller 301 described above with respect to FIG. 3. In various embodiments, at block 902, the controller is configured to request and collect a passenger's consent. Responsive to the passenger providing their consent, at block 904, the controller is configured to collect circadian data from the passenger. At block 906, the controller is configured to request the passenger's consent to adjust cabin conditions based on their circadian rhythm for a specific trip. At block 908, the controller is configured to receive data from passenger wearable devices or other sources to through cabin Wi-Fi, Bluetooth, etc. At block 910, the controller is configured to analyze personal and circadian data to recognize patterns and establish a circadian rhythm model for the passenger. At block 912, the controller is configured to establish communications/integration with devices associated with the cabin management system, flight control system, or other sensors associated with the suite, among other. At block 914, the controller is configured to perform automatic cabin adjustments for passenger circadian comfort using the circadian model. At block 916, the controller is configured to survey the passenger at the end of the flight for passenger feedback, which may be utilized in the post-flight analysis. At block 918, the controller is configured to perform post-flight analysis in order to review the collected data to assess the effectiveness of the adjustments made during the flight. The post-flight analysis may analyze passenger feedback, circadian data, and other relevant parameters to evaluate the overall comfort level and effectiveness of the system in mitigating jet lag and enhancing passenger well-being. In various embodiments, the post-flight analysis may assist in identifying areas for improvement and refine the system for future flights.

In various embodiments, the system and method for adjusting suite conditions based on circadian rhythms of a passenger may provide for one or more of enhanced comfort, reduced jet lag, improved well-being, a personalized experience, increased productivity, or enhanced recovery, among others. In various embodiments, the enhanced comfort may be provided by aligning cabin conditions with passenger's natural circadian rhythms, ensuring a more comfortable and restful travel experience. In various embodiments, the reduced jet lag may be provided by dynamic adjusting cabin conditions based on circadian rhythms helps minimize jet lag symptoms, enabling passengers to adapt more seamlessly to new time zones. In various embodiments, the improved well-being may be provided by optimizing cabin conditions to support circadian rhythms, thereby contributing to the passenger's overall well-being and reduces stress associated with air travel. In various embodiments, the personalized experience may be provided by the system's ability to adapt to individual circadian patterns allows for a personalized in-flight experience tailored to each passenger's preferences and needs. In various embodiments, the increased productivity may be provided by promoting better sleep and reducing fatigue, enabling the passenger to arrive at their destination feeling more alert and focused, enhancing productivity upon arrival. In various embodiments, the enhanced recovery may be provided through optimal cabin conditions supporting the passenger's post-flight recovery and minimizing the impact of travel-related fatigue and allowing for a smoother transition to their destination.

System program instructions and/or controller instructions may be loaded onto a non-transitory, tangible computer-readable medium having instructions stored thereon that, in response to execution by a controller, cause the controller to perform various operations. The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

For example, the steps recited in any of the method or process descriptions may be executed in any order and are not limited to the order presented. Moreover, any of the functions or steps may be outsourced to or performed by one or more third parties. Modifications, additions, or omissions may be made to the systems, apparatuses, and methods described herein without departing from the scope of the disclosure. For example, the components of the systems and apparatuses may be integrated or separated. An individual component may be comprised of two or more smaller components that may provide a similar functionality as the individual component. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Furthermore, any reference to singular includes plural embodiments, and any reference to more than one component may include a singular embodiment. Use of ‘a’ or ‘an’ before a noun naming an object shall indicate that the phrase be construed to mean ‘one or more’ unless the context sufficiently indicates otherwise, as set forth in Slip op. at 8-9 (Fed. Cir. Oct. 19, 2023) (citing Baldwin Graphic Sys., Inc. v. Siebert, Inc., 512 F.3d 1338, 1342-43 (Fcd. Cir. 2008)). For example, the description or claims may refer to a processor for convenience, but the invention and claim scope contemplates that the processor may be multiple processors. The multiple processors may manage separate tasks or combine to manage certain tasks. Although specific advantages have been enumerated herein, various embodiments may include some, none, or all of the enumerated advantages. A “processor” may include hardware that runs the computer program code. Specifically, the term ‘processor’ may be synonymous with terms like controller and computer and should be understood to encompass not only computers having different architectures such as single/multi-processor architectures and sequential (Von Neumann)/parallel architectures but also specialized circuits such as field-programmable gate arrays (FPGA). application specific circuits (ASIC), signal processing devices and other devices.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C. Different cross-hatching is used throughout the figures to denote different parts but not necessarily to denote the same or different materials.

Systems, methods, and apparatus are provided herein. In the detailed description herein, references to “one embodiment,” “an embodiment,” “various embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Numbers, percentages, or other values stated herein are intended to include that value, and also other values that are about or approximately equal to the stated value, as would be appreciated by one of ordinary skill in the art encompassed by various embodiments of the present disclosure. A stated value should therefore be interpreted broadly enough to encompass values that are at least close enough to the stated value to perform a desired function or achieve a desired result. The stated values include at least the variation to be expected in a suitable industrial process, and may include values that are within 5% of a stated value. Additionally, the terms “substantially,” “about” or “approximately” as used herein represent an amount close to the stated amount that still performs a desired function or achieves a desired result. For example, the term “substantially,” “about” or “approximately” may refer to an amount that is within 5% of a stated amount or value.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Finally, it should be understood that any of the above-described concepts can be used alone or in combination with any or all of the other above-described concepts. Although various embodiments have been disclosed and described, one of ordinary skill in this art would recognize that certain modifications would come within the scope of this disclosure. Accordingly, the description is not intended to be exhaustive or to limit the principles described or illustrated herein to any precise form. Many modifications and variations are possible in light of the above teaching.

What is claimed is:

1. A suite management system, the suite management system comprising:
a controller, the controller configured to:
obtain passenger preferences from a passenger occupying a suite;
obtain circadian data from the passenger;
analyze the circadian data to identify one or more changes to the suite to align with the passenger preferences; and
responsive to identifying the one or more changes, automatically adjust one or more electronic devices within the suite to align with the passenger preferences.

2. The suite management system of claim 1, wherein the circadian data comprises at least one of a heart rate, body temperature, sleep patterns, blood oxygen saturation levels, or a heart rate variability (HRV) of the passenger.

3. The suite management system of claim 2, wherein the circadian data further comprises at least one of respiratory rate, skin temperature gradient, electrodermal activity, cognitive load, gastrointestinal activity, hydration, mood and emotional state, movement, activity level, sleep quality, allergy and air quality, thermal comfort, blood glucose, A1C, posture, pressure points, vibration or balance.

4. The suite management system of claim 1, wherein the circadian data is obtained from at least one of a smartwatch, a fitness tracker, an activity tracker, or a phone of the passenger.

5. The suite management system of claim 4, wherein the circadian data is obtained at least one sensor coupled to the at least one of the smartwatch, the fitness tracker, the activity tracker, or the phone of the passenger.

6. The suite management system of claim 5, wherein the at least one sensor comprises at least one of a respiratory sensor, a heart rate sensor, a skin temperature sensor, a electrodermal activity sensor, a smart patch or ingestible sensor, an electroencephalogram (EEG) sensor, a voice analysis sensor, a facial expression sensor, a hydration tracking sensor, an oxygen saturation (SpO₂) sensor, an environmental sensor, a continuous glucose monitoring (GCM) sensor, an A1C sensor, a posture sensor, a pressure point mapping sensor, an accelerometer sensor, or a gyroscope sensor.

7. The suite management system of claim 1, wherein the one or more electronic devices comprise at least one of a light, a seat, a temperature control system, a flight attendant call system, or an in-flight entertainment system.

8. The suite management system of claim 7, wherein the one or more electronic devices further comprise at least one of an oxygen supply system, an air circulation system, a humidity control system, an air purifier, or an air filtration system.

9. The suite management system of claim 1, wherein the controller is at least one of electronically or communicatively coupled to an electrical system or a communication system in order to adjust the one or more electronic devices within the suite.

10. The suite management system of claim 1, wherein the controller is further configured to:
analyze the circadian data to identify at least one of a suggestion, a reminder, or an alert send to the passenger; and
responsive to identifying the at least one of the suggestion, the reminder, or the alert, automatically send the at least one of the suggestion, the reminder, or the alert to the passenger.

11. An aircraft, the aircraft comprising:
a suite; and
a suite management system deployed within the suite, the suite management system comprising:
a controller, the controller configured to:
obtain passenger preferences from a passenger occupying the suite;
obtain circadian data from the passenger;
analyze the circadian data to identify one or more changes to the suite to align with the passenger preferences; and
responsive to identifying the one or more changes, automatically adjust one or more electronic devices within the suite to align with the passenger preferences.

12. The aircraft of claim 11, wherein the circadian data comprises at least one of a heart rate, body temperature, sleep patterns, oxygen saturation levels, or a heart rate variability (HRV) of the passenger.

13. The aircraft of claim 12, wherein the circadian data further comprises at least one of respiratory rate, skin temperature gradient, electrodermal activity, cognitive load, gastrointestinal activity, hydration, mood and emotional state, movement, activity level, sleep quality, allergy and air quality, thermal comfort, blood glucose, A1C, posture, pressure points, vibration or balance.

14. The aircraft of claim 11, wherein the circadian data is obtained from at least one of a smartwatch, a fitness tracker, an activity tracker, or a phone of the passenger.

15. The aircraft of claim 14, wherein the circadian data is obtained at least one sensor coupled to the at least one of the smartwatch, the fitness tracker, the activity tracker, or the phone of the passenger.

16. The aircraft of claim 15, wherein the at least one sensor comprises at least one of a respiratory sensor, a heart rate sensor, a skin temperature sensor, a electrodermal activity sensor, a smart patch or ingestible sensor, an electroencephalogram (EEG) sensor, a voice analysis sensor, a facial expression sensor, a hydration tracking sensor, an oxygen saturation (SpO₂) sensor, an environmental sensor, a continuous glucose monitoring (GCM) sensor, an A1C sensor, a posture sensor, a pressure point mapping sensor, an accelerometer sensor, or a gyroscope sensor.

17. The aircraft of claim 11, wherein the one or more electronic devices comprise at least one of a light, a seat, a temperature control system, a flight attendant call system, or an in-flight entertainment system.

18. The aircraft of claim 17, wherein the one or more electronic devices further comprise at least one of an oxygen supply system, an air circulation system, a humidity control system, an air purifier, or an air filtration system.

19. The aircraft of claim 11, wherein the controller is at least one of electronically or communicatively coupled to an electrical system or a communication system in order to adjust the one or more electronic devices within the suite.

20. The aircraft of claim 11, wherein the controller is further configured to:
analyze the circadian data to identify at least one of a suggestion, a reminder, or an alert send to the passenger; and
responsive to identifying the at least one of the suggestion, the reminder, or the alert, automatically send the at least one of the suggestion, the reminder, or the alert to the passenger.
